(19) United States
(12) Patent Application Publication
(10) Pub. No.: US 2022/0008356 A1
(43) Pub. Date: Jan. 13, 2022

(54) TREATMENT OF SKIN DISORDERS WITH TOPICAL TAPINAROF COMBINATION COMPOSITIONS

(71) Applicant: Sol-Gel Technologies Ltd., Ness Ziona (IL)

(72) Inventor: Moshe ARKIN, Kfar Shmaryahu (IL)

(73) Assignee: Sol-Gel Technologies Ltd., Ness Ziona (IL)

(21) Appl. No.: 17/425,956

(22) PCT Filed: Jan. 27, 2020
(86) PCT No.: PCT/IL2020/050103

§ 371 Date: Jul. 27, 2021

Related U.S. Application Data
(60) Provisional application No. 62/797,298, filed on Jan. 27, 2019.

(51) Int. Cl. A61K 31/05; A61K 9/00; A61K 31/573; A61K 31/593; A61K 9/06; A61K 31/519

(52) U.S. Cl. CPC A61K 31/05; A61K 9/0014; A61K 31/519; A61K 31/593; A61K 9/06; A61K 31/573

(57) ABSTRACT

Provided herein are topical combination compositions comprising a therapeutically effective dose of tapinarof and a therapeutically effective dose of at least one additional active agent, selected from at least one corticosteroid, calcipotriene, at least one JAK inhibitor and combinations thereof, and a carrier suitable for topical administration. The above compositions are useful for the treatment, prevention or alleviation of skin disorders selected from psoriasis and atopic dermatitis and exhibit synergistic or additive effects which allow reducing the doses of the active agents in the compositions.

### FIELD OF THE INVENTION

The present invention, in some embodiments thereof, relates to treatment of skin disorders by topical administration of combination compositions comprising tapinarof and at least one additional active agent. The compositions of this invention are useful for the treatment, prevention or amelioration of skin disorders and exhibit synergistic or additive effects which allow reducing the dose of the active agents in the combination composition.

### BACKGROUND OF THE INVENTION

Tapinarof (3,5-dihydroxy-4-isopropyl-trans-stilbene) is a pharmaceutical active agent investigated for the treatment of atopic dermatitis, psoriasis and psoriatic disorders (Zang Y N, et al., Int J Clin Pharmacol Ther. 2016 February; 54(2): 87-95). The 3,5-dihydroxy-4-isopropyl-stilbene is also known as benvitimod.

Tapinarof is a first-in-class drug, whose mechanism is not yet fully understood.

Tapinarof is being developed by Glaxo Smith Kline (Stiefel, a GSK company) and Dermavant as a topical drug for treatment of mild to moderate plaque psoriasis and atopic dermatitis. It was shown in both mouse models and in vitro human skin studies to inhibit specific proinflammatory mediators, including interleukin-6 and interleukin-17A, and enhance skin barrier function (J Invest Dermatol. 2017 October; 137[10]:2110-9).

A number of GSK28994512 (tapinarof) clinical studies were completed:
1. Dose finding study (Phase I) of GSK28994512 in subjects with plaque psoriasis—1% (10 mg/g) and 0.5% (5 mg/g) creams for two application frequencies, once a day and twice a day, vs. vehicle cream.
2. Dose finding study of GSK28994512 (Phase II) in subjects with atopic dermatitis—1% (10 mg/g) and 0.5% (5 mg/g) creams for two application frequencies, once a day and twice a day, vs. vehicle cream.
3. Dose finding study (Phase II) of GSK28994512 in subjects with plaque psoriasis—1% (10 mg/g) and 0.5% (5 mg/g) creams for two application frequencies, once a day and twice a day, vs. vehicle cream.
4. Skin irritation study (Phase I) of GSK28994512 cream (0.5% and 1%) vs vehicle cream.
5. A single dose (Phase I) exploratory study in healthy volunteers with GSK28994512 cream for atopic dermatitis. Two cream formulations (GSK2894512 cream A and GSK2894512 cream B) were tested in combination for skin residency.
6. Pharmacokinetic study (Phase I) of topical GSK2894512 1% and 2% creams following twice daily application of the 2% cream (cohort 1) or 1% cream (cohort 2).

According to Jancin B. (Dermatology News, Nov. 11, 2017), in the above studies, the 1% tapinarof arm showed higher efficacy and had a quicker onset of action than the 0.5% arm or vehicle. The most frequent adverse events associated with tapinarof were folliculitis and contact dermatitis.

Tapinarof, which seems to be a significant advance in psoriasis treatment, presents however higher adverse effects (44.5%) when compared to placebo (20.2%) and calcipotriene (19.5%) as a single drug (Medscape Mar. 5, 2017 report by Frellick M., on Abstr. 5629, Amer. Acad. of Dermatology meeting, Mar. 4, 2017).

Psoriasis is an autoimmune disease, characterized by typically red, scaly patches of skin. The following main types of psoriasis include: plaque, guttate, inverse, pustular, flexural/inverse and erythrodermic. Plaque psoriasis (psoriasis vulgaris) is the most common form of psoriasis.

Inverse psoriasis is a rare form of psoriasis which is also known as flexural or intertriginous psoriasis. This subtype of psoriasis can occur in any area where two skin surfaces meet. Classically the skin of the groin region, armpits and genitals are affected. In these regions the skin appears red, shiny, and moist, with clear borders, and can sometimes crack in the centre.

Some of the known topical psoriasis treatments use pharmaceutical active agents like corticosteroids like desoxymethasone and Vitamin D analogues like calcipotriene or paricalcitol. Combinations of several of the above classes of active agents, like Vitamin D and corticosteroids have been investigated. Most of the known psoriasis topical treatments in general, and those comprising steroids in particular, present undesirable side-effects.

Though a number of psoriasis treatments are available, most treatments bring about symptom alleviation or remission rather than complete cure.

There is an unmet need for methods for the treatment of skin disorders using tapinarof topical compositions, devoid of serious side-effects.

### SUMMARY OF THE INVENTION

This invention provides a topical composition comprising from about 0.25% w/w to about 2.0% w/w tapinarof and at least one additional active agent, selected from about 0.01% w/w to about 0.25% w/w at least one corticosteroid of potency class 1 or 2 or from about 0.0025% w/w to about 2.5% w/w at least one corticosteroid of potency class 3-7, from about 0.001% w/w to about 0.005% w/w calcipotriene, from about 0.1% w/w to about 3% w/w at least one JAK1, JAK2 or JAK 1/2 inhibitor and combinations thereof, and a carrier suitable for topical administration.

The composition is suitable for the treatment, prevention or amelioration of skin disorders selected from psoriasis and atopic dermatitis, and exhibit synergistic or additive effects which allow reducing the dose of the active agents in the combination compositions.

### DETAILED DESCRIPTION OF THE INVENTION

The present invention provides novel methods of treatment and topical compositions useful for the treatment, prevention and amelioration and of skin disorders selected from psoriasis and atopic dermatitis.

In some embodiment, the present invention provides a topical compositions comprising tapinarof and at least one additional active agent selected from at least one corticosteroid, Vitamin D analogue, at least one JAK inhibitor and combinations thereof, wherein the combination composition exhibits improved therapeutic effects and also synergistic or additive effects in the treatment of skin disorders selected from psoriasis and atopic dermatitis and, as a result, allow using lower dosage of the actives and alleviate the side-effects (like local irritation and contact dermatitis).

In another embodiment the Vitamin D analogue is calcipotriene, paricalcitol or hydrate thereof. In another embodiment, the Vitamin D analogue is calcipotriene or hydrate thereof. In another embodiment, the Vitamin D analogue is paricalcitol or hydrate thereof.

The tapinarof combination compositions of this invention have a double advantage vs. the use of tapinarof as a single drug: on the one hand the at least one additional active has the potential to alleviate the tapinarof side-effects and on the other hand the synergistic or additive effect enables using lower active agent dosages.

### Corticosteroids

The at least one corticosteroid is selected from steroids of various potencies (see below Class 1-Class 7), approved and marketed in the US for topical use.

### Topical Steroid Classification by Potency

According to the “Topical steroid potency chart” of the National Psoriasis Foundation (NPF), the various marketed topical drugs comprising steroids belong to the following potency classes, according to the steroid and the topical drug strength. Due to the different topical drug strength, drugs of different strengths and/or different dosage forms may belong to more than one steroid class. The percentages in parentheses are the steroid strengths for the FDA-approved topical steroid compositions.

Class 1—superpotent, comprising 7 steroids: clobetasol propionate (0.05%), flurandrenolide (0.05%), betamethasone dipropionate (0.05%), diflorasone diacetate (0.05%), desoxymethasone, and fluocinonide (0.1%).

Class 2—potent, comprising 6 steroids: betamethasone dipropionate (0.05%), mometasone furoate (0.1%), diflorasone diacetate (0.05%), halcinonide (0.1%), fluocinonide (0.05%), desoxymethasone (0.05%-0.25%).

Class 3—upper mid-strength, comprising 3 steroids: fluticasone propionate (0.005%), fluocinonide (0.05%) and betamethasone valerate (0.12%).

Class 4—mid-strength, comprising 6 steroids: flurandrenolide (0.05%), mometasone furoate (0.1%), triamcinolone acetonide (0.1%), fluocinolone acetonide (0.03%), desoxymethasone (0.05%) and hydrocortisone valerate (0.2%).

Class 5—lower mid-strength, comprising 7 steroids: fluocinolone acetonide (0.01%), flurandrenolide (0.05%), fluticasone propionate (0.05%), prednicarbate (0.1%), desonide (0.05%), hydrocortisone (0.1%), hydrocortisone valerate (0.2%).

Class 6—mild, comprising only 3 steroids: alclomethasone dipropionate (0.05%), fluocinolone acetonide (0.01%), desonide (0.05%),

Class 7—least potent, comprising only one steroid: hydrocortisone (0.5%, 1%, 2%, 2.5%).

The above corticosteroids are marketed as lotions, creams, solutions, ointments, foam, sprays, gels. Many of the above topical drugs are sold as creams.

### Calcipotriene

Calcipotriene (also known as calcipotriol) is used inter alia. in the topical treatment of several topical disorders, including psoriasis.

Topical calcipotriene and calcipotriene hydrate drugs are FDA-approved as 0.005% solution, cream and aerosol foam dosage forms.

Combinations of 0.005% calcipotriene with 0.064% betamethasone dipropionate are FDA-approved as ointment, suspension and aerosol foam.

### JAK Inhibitors

JAK (Janus kinase) inhibitors (JAKi), also known as jakinibs, are drugs inhibiting the activity of one or more of the Janus kinase family of enzymes (JAK1, JAK2, JAK 1/2, JAK3, TYK2). JAK3 inhibitors have been investigated for the treatment of autoimmune diseases.

Selective JAK3 inhibitors are being developed (Forster M, et al. (September 2017). “Recent advances in JAK3 inhibition: Isoform selectivity by covalent cysteine targeting”. Bioorganic & Medicinal Chemistry Letters. 27 (18): 4229-4237).

A JAK1 inhibitor (PF-04965842, Pfizer) is investigated for treatment of atopic dermatitis and moderate to severe psoriasis (ClinicalTrials.gov).

In some embodiments, baricitithb is a JAK 1/2 inhibitor.

In some embodiment, the at least one JAK inhibitor in the compositions of this invention is selected from a JAK1 inhibitor, a JAK2 inhibitor, a JAK 1/2 inhibitor, a JAK3 inhibitor, a TYK2 inhibitor and combinations thereof.

In some embodiments, there is provided any one of the compositions of this invention, wherein said at least one JAK inhibitor is a pan-JAK inhibitor.

In some embodiments, there is provided any one of the methods of treatment of this invention, wherein said at least one JAK inhibitor is a pan-JAK inhibitor.

In some embodiments the at least one JAK inhibitor is selected from tofacitinib, abrocitinib, ruxolitinib, delgocitinib, oclacitinib, baricitinib, peficitinib and combinations thereof.

The tapinarof/JAKi or tapinarof/JAKi/corticosteroid synergistic or additive effect of the compositions of this invention allows using lower doses of JAK inhibitors and the topical route avoids systemic side-effects on the immune system due to absorption of potentially toxic JAK inhibitors.

### Topical Tapinarof Combination Compositions

Provided herein are compositions, combinations, kits and articles of manufacture that include tapinarof in combination with at least one additional active agent, for treating a skin disorder selected from psoriasis and atopic dermatitis. The compositions, combinations and articles of manufacture can be administered using a variety of routes such as topical application or transdermal application. As one example, provided herein is an article of manufacture that includes tapinarof, at least one additional active agent and a carrier for topical administration, for treating a skin disorder selected from psoriasis and atopic dermatitis.

Also provided herein is an article of manufacture that includes tapinarof and at least one additional active agent, adjusted to a dosage suitable for the treatment of a skin disorder, and a carrier for topical administration. The articles of manufacture provided herein can further contain a label indicating that the composition is for treating a skin disorder as provided herein. The at least one additional active agent may be at least one corticosteroid of a strength suitable for the medical indication, vitamin D analogues, at least one JAK inhibitor or combinations thereof.

In another embodiment the Vitamin D analogue is calcipotriene, paricalcitol or hydrate thereof. In another embodiment, the Vitamin D analogue is calcipotriene or hydrate thereof. In another embodiment, the Vitamin D analogue is paricalcitol or hydrate thereof.

The at least one corticosteroid in the compositions for the treatment of psoriasis may be superpotent (Class 1) or potent (Class 2). Alternatively, the steroid may be of lower potency (Class 3-7), thus minimizing the steroid side-effects, including the risk of pituitary suppression.

The at least one corticosteroid in the compositions for the treatment of atopic dermatitis may be of medium or low potency (Class 4-7).

The articles of manufacture provided herein can further include a delivery system.

The delivery system can be selected from among a variety of vehicles for administering therapeutic agents, as known to those of skill in the art. For example, the delivery system can be selected from among a transdermal patch, a lotion, a cream, an ointment, a gel, a spray, a foam delivery system or an applicator syringe.

Also provided herein are compositions containing tapinarof and at least one additional active agent selected from at least one corticosteroid, vitamin D analogues (such as calcipotriene), at least one JAK inhibitor and combinations thereof and a carrier suitable for topical administration.

The at least one corticosteroid can be selected from among any of those provided herein, incorporated by reference herein, identified by assays as provided herein, or known to those of skill in the art.

Therapeutically effective concentrations for treatment, prevention or amelioration of the symptoms manifested by the skin disorder of tapinarof and at least one additional active agent is mixed with a suitable pharmaceutical carrier or vehicle for topical, transdermal or other routes.

Tapinarof and the at least one additional active agent in the combination compositions are included in an amount effective for reducing the skin disorder for which treatment is contemplated. The concentration of the active agents in the composition will depend on absorption, inactivation, excretion rates of the active compound, the synergistic or additive effects, the dosage schedule, and amount administered as well as other factors known to those of skill in the art. Generally, the dosages and concentrations will be lower, typically at least about or at 5 to 10% lower but up to about or at 15, 20, 25, 30, 35, 40, 50, 90 or 95% lower than the amount of tapinarof and/or the at least one additional active agent in the marketed drug currently administered for the treatment of the contemplated skin disorder. The dosage and regimen of administration may be determined by dose finding studies, as known in the art.

Exemplary dosages, strengths and concentrations of tapinarof in the tapinarof combination compositions administered topically, can be in the range of from about or at 0.1%, 0.25%, 0.5%, 1%, 2% or 3% w/w. Typical strengths in the topical combination compositions of this invention are 0.25%, 0.5% or 1% w/w tapinarof. The frequency of administration can be determined empirically. Exemplary frequencies are once daily, twice daily, weekly, bi-weekly or monthly. Typical administration frequencies of the topical combination compositions of this invention are once daily and twice daily.

Dosage frequencies can be gradually attenuated over time and maintained at a steady dose suitable for long-term—six months, 1 year, 5 years, 10 years or more, up to lifelong administration to control the symptoms of the skin disorder. For example, dosage administration can begin at from twice a day, to once a day, to two times a week, to once a week, to once every two weeks or less frequent than once every two weeks.

Pharmaceutical carriers or vehicles suitable for preparation of the compositions provided herein include any such carriers known to those skilled in the art to be suitable for the particular mode of administration.

The resulting compositions may be a solution, suspension, emulsion or the like and are formulated as creams, gels, ointments, emulsions, solutions, elixirs, lotions, suspensions, tinctures, pastes, foams, aerosols, sprays, patches, foams or any other formulation suitable for topical administration.

Pharmaceutical carriers or vehicles suitable for administration of the compounds provided herein include any such carriers known to those skilled in the art to be suitable for the particular mode of administration. In addition, the compounds may be formulated as the sole pharmaceutically active ingredient in the composition or may be combined with other active ingredients. The active agents are included in the carrier in an amount sufficient to exert a therapeutically useful effect i.e., amelioration of the symptoms of the skin disorder, with minimal or no toxicity or other side effects. Generally, emollient or lubricating vehicles that help hydrate the skin are more preferred than volatile vehicles, such as ethanol, that dry the skin. Examples of suitable bases or vehicles for preparing compositions for use with human skin are petrolatum, petrolatum plus volatile silicones, lanolin, cold cream and hydrophilic ointment.

Suitable pharmaceutically and dermatologically acceptable vehicles for topical application include those suited for use include lotions, creams, solutions, gels, tapes and the like. Generally, the vehicle is either organic in nature or an aqueous emulsion and capable of having the selected compound or compounds, which may be micronized, dispersed, suspended or dissolved therein. The vehicle may include pharmaceutically-acceptable emollients, moisturizers, including lactic acid, ammonium lactate and urea, skin penetration enhancers, coloring agents, fragrances, emulsifiers, thickening agents, vegetable oils, essential oils, zinc oxide and solvents.

### Tapinarof Combination Compositions for the Treatment of Psoriasis

The at least one corticosteroid in the compositions of this invention for the treatment, prevention or amelioration of psoriasis may be superpotent (Class 1) or potent (Class 2). Alternatively, the steroid may be of lower potency, selected from upper mid-strength (Class 3), mid-strength (Class 4) lower mid-strength (Class 5), mild (Class 6) or least potent (Class 7), thus minimizing the steroid side-effects, including the risk of pituitary suppression.

The superpotent (Class 1) or potent (Class 2) corticosteroids are selected from the following steroids (The percentages in parentheses are the steroid strengths for the FDA-approved topical steroid compositions):

Class 1—superpotent, comprising 7 steroids: clobetasol propionate (0.05%), flurandrenolide (0.05%), betamethasone dipropionate (0.05%), diflorasone diacetate (0.05%), desoxymethasone, and fluocinonide (0.1%).

Class 2—potent, comprising 6 steroids: betamethasone dipropionate (0.05%), mometasone furoate (0.1%), diflorasone diacetate (0.05%), halcinonide (0.1%), fluocinonide (0.05%), desoxymethasone (0.05%-0.25%).

The lower potency steroids are selected from:

Class 3—upper mid-strength, comprising 3 steroids: fluticasone propionate (0.005%), fluocinonide (0.05%) and betamethasone valerate (0.12%).

Class 4—mid-strength, comprising 6 steroids: flurandrenolide (0.05%), mometasone furoate (0.1%), triamcinolone acetonide (0.1%), fluocinolone acetonide (0.03%), desoxymethasone (0.05%) and hydrocortisone valerate (0.2%).

Class 5—lower mid-strength, comprising 7 steroids: fluocinolone acetonide (0.01%), flurandrenolide (0.05%), fluticasone propionate (0.05%), prednicarbate (0.1%), desonide (0.05%), hydrocortisone (0.1%), hydrocortisone valerate (0.2%).

Class 6—mild, comprising only 3 steroids: alclomethasone dipropionate (0.05%), fluocinolone acetonide (0.01%), desonide (0.05%).

Class 7—least potent, comprising only one steroid: hydrocortisone (0.5%, 1%, 2%, 2.5%).

In some embodiments, there is provided a combination composition for the treatment, prevention or amelioration of psoriasis, (including plaque psoriasis, or flexural/inverse psoriasis) by topical administration to a subject in need thereof of a composition comprising a therapeutically effective dose of tapinarof and a therapeutically effective dose of at least one additional active agent selected from at least one superpotent (Class 1) or potent (Class 2) corticosteroid, calcipotriene and combinations thereof and a carrier suitable for topical administration.

According to some embodiments, the above at least one superpotent (Class 1) corticosteroid is selected from (the percentages in parentheses are the steroid strengths for the FDA-approved topical steroid compositions) clobetasol propionate (0.05%), flurandrenolide (0.05%), betamethasone dipropionate (0.05%), diflorasone diacetate (0.05%), desoxymethasone, and fluocinonide (0.1%) and combinations thereof and the potent (Class 2) corticosteroid is selected from betamethasone dipropionate (0.05%), mometasone furoate (0.1%), diflorasone diacetate (0.05%), halcinonide (0.1%), fluocinonide (0.05%), desoxymethasone (0.05%-0.25%) and combinations thereof.

In some other embodiments, there is provided a combination composition for the treatment, prevention or amelioration of psoriasis, (including plaque psoriasis, or flexural/inverse psoriasis) by topical administration to a subject in need thereof of a composition comprising a therapeutically effective dose of tapinarof and a therapeutically effective dose of at least one additional active agent selected from at least one corticosteroid of mid-strength (Class 4), lower mid-strength (Class 5), mild (Class 6) or least potent (Class 7), calcipotriene, combinations thereof and a carrier suitable for topical administration.

According to some embodiments, there is provided a combination composition for the treatment, prevention or amelioration of psoriasis, (including plaque psoriasis, or flexural/inverse psoriasis) by topical administration to a subject in need thereof of a composition comprising a therapeutically effective dose of tapinarof, a therapeutically effective dose of at least one corticosteroid of mid-strength (Class 4), lower mid-strength (Class 5), mild (Class 6) or least potent (Class 7), a therapeutically effective dose of calcipotriene, combinations thereof and a carrier suitable for topical administration.

According to some other embodiments, there is provided a combination composition for the treatment, prevention or amelioration of psoriasis, (including plaque psoriasis or flexural/inverse psoriasis) by topical administration to a subject in need thereof of a composition comprising a therapeutically effective dose of tapinarof and a therapeutically effective dose of at least one corticosteroid of mid-strength (Class 4), lower mid-strength (Class 5), mild (Class 6) or least potent (Class 7), combinations thereof and a carrier suitable for topical administration.

In some other embodiments, the strength of the at least one corticosteroid in the compositions for the treatment, prevention or amelioration of psoriasis is 25% w/w, 50% w/w or 75% w/w lower that the strength of the FDA-approved corticosteroids (see above FDA-approved strengths in parentheses).

In some embodiments, there is provided a composition for the treatment, prevention or amelioration of psoriasis, comprising 0.25-2% w/w tapinarof, 0.01-0.05% w/w betamethasone dipropionate and a carrier suitable for topical administration.

In some embodiments, there is provided a composition for the treatment, prevention or amelioration of psoriasis, comprising 0.5% w/w tapinarof, 0.025% w/w betamethasone dipropionate and a carrier suitable for topical administration.

In some embodiments, there is provided a composition for the treatment, prevention or amelioration of psoriasis, comprising 0.25-2% w/w tapinarof, 0.001-0.005% w/w calcipotriene and a carrier suitable for topical administration.

In some embodiments, there is provided a composition for the treatment, prevention or amelioration of psoriasis, comprising 0.5% w/w tapinarof, 0.0025% w/w calcipotriene and a carrier suitable for topical administration.

In some embodiments, there is provided a composition for the treatment, prevention or amelioration of psoriasis, comprising 0.25-2% w/w tapinarof, 0.001-0.005% w/w calcipotriene, 0.01-0.05% betamethasone dipropionate and a carrier suitable for topical administration.

In some embodiments, there is provided a composition for the treatment, prevention or amelioration of psoriasis, comprising 0.5% w/w tapinarof, 0.0025% w/w calcipotriene, 0.025% betamethasone dipropionate and a carrier suitable for topical administration.

The above composition may be a solution, suspension, emulsion or the like and is formulated as a cream, a gel, an ointment, an emulsion, a solution, an elixir, a lotion, a suspension, a tincture, a paste, a foam, an aerosol, a spray, a patch or any other formulation suitable for topical administration.

In some embodiments the composition described herein for the treatment, prevention or amelioration of psoriasis include the main types of psoriasis: plaque, guttate, inverse, pustular, flexural/inverse and erythrodermic. Plaque psoriasis (psoriasis vulgaris) is the most common form of psoriasis.

### Tapinarof Combination Compositions for the Treatment of Atopic Dermatitis

According to some embodiments, there is provided a composition for the treatment, prevention or amelioration of atopic dermatitis, comprising a therapeutically effective dose of tapinarof in combination with a therapeutically effective dose of at least one additional active agent selected from of at least one medium or low potency corticosteroid (Class 4-7), at least one JAK inhibitor, combinations thereof and a carrier suitable for topical administration.

In some embodiments, there is provided a composition for the treatment, prevention or amelioration of atopic dermatitis, comprising a therapeutically effective dose of tapinarof, a therapeutically effective dose of at least one medium or low potency corticosteroid (Class 4-7) and a carrier suitable for topical administration.

In some other embodiments, there is provided a composition for the treatment, prevention or amelioration of atopic dermatitis, comprising a therapeutically effective dose of tapinarof in combination with a therapeutically effective dose of a therapeutically effective dose of a JAK inhibitor.

In some other embodiments, there is provided a composition for the treatment, prevention or amelioration of atopic dermatitis, comprising a therapeutically effective dose of tapinarof, a therapeutically effective dose of at least one medium or low potency corticosteroid (Class 4-7), a therapeutically effective dose of a JAK1 or a JAK2 inhibitor and a carrier suitable for topical administration.

In some embodiments, there is provided a composition for the treatment, prevention or amelioration of atopic dermatitis, comprising 0.25-2% w/w tapinarof, 0.05-0.2% w/w triamcinolone and a carrier suitable for topical administration.

In some other embodiments, there is provided a composition for the treatment, prevention or amelioration of atopic dermatitis, comprising 0.5% w/w tapinarof, 0.1% w/w triamcinolone and a carrier suitable for topical administration.

According to some embodiments, there is provided a composition for the treatment, prevention or amelioration of atopic dermatitis, comprising 0.25-2% w/w tapinarof, 1-2% w/w ruxolitinib phosphate (calculated as base) and a carrier suitable for topical administration.

In some embodiments, there is provided a composition for the treatment, prevention or amelioration of atopic dermatitis, comprising 0.5% w/w tapinarof, 1.5% w/w ruxolitinib phosphate (calculated as base) and a carrier suitable for topical administration.

In some other embodiments, there is provided a composition for the treatment, prevention or amelioration of atopic dermatitis, comprising 0.25-2% w/w tapinarof, 0.05-0.2% w/w triamcinolone, 1-2% w/w ruxolitinib phosphate (calculated as base) and a carrier suitable for topical administration.

Other exemplary compositions of this invention are detailed in Examples 1-6.

According to some embodiments, there is provided a composition for the treatment, prevention or amelioration of atopic dermatitis, comprising 0.5% w/w tapinarof, 0.1% w/w triamcinolone, 1.5% w/w ruxolitinib and a carrier suitable for topical administration.

According to some other embodiments, there is provided a composition for the treatment, prevention or amelioration of atopic dermatitis, comprising 0.5% w/w tapinarof, 0.05% w/w triamcinolone, 0.75% w/w ruxolitinib and a carrier suitable for topical administration.

The above composition may be a solution, suspension, emulsion or the like and is formulated as a cream, a gel, an ointment, an emulsion, a solution, an elixir, a lotion, a suspension, a tincture, a paste, a foam, an aerosol, a spray, a patch or any other formulation suitable for topical administration.

The above Class 4-7 corticosteroids are selected from the following steroids (FDA-approved strengths are indicated in parentheses).

Class 4—mid-strength, comprising 6 steroids: flurandrenolide (0.05%), mometasone furoate (0.1%), triamcinolone acetonide (0.1%), fluocinolone acetonide (0.03%), desoxymethasone (0.05%) and hydrocortisone valerate (0.2%).

Class 5—lower mid-strength, comprising 7 steroids: fluocinolone acetonide (0.01%), flurandrenolide (0.05%), fluticasone propionate (0.05%), prednicarbate (0.1%), desonide (0.05%), hydrocortisone (0.1%), hydrocortisone valerate (0.2%).

Class 6—mild, comprising only 3 steroids: alclomethasone dipropionate (0.05%), fluocinolone acetonide (0.01%), desonide (0.05%),

Class 7—least potent, comprising only one steroid: hydrocortisone (0.5%, 1%, 2%, 2.5%).

In some other embodiments, the strength of the at least one corticosteroid in the compositions for the treatment, prevention or amelioration of atopic dermatitis is 25% w/w, 50% w/w or 75% w/w lower that the strength of the FDA-approved corticosteroids (see above FDA-approved strengths in parentheses).

### Methods of Treatment

According to an aspect of the invention, there is provided a method of treatment, prevention or amelioration of a skin disorder which is treatable, preventable and/or alleviated by treatment of a subject in need thereof with a combination composition of tapinarof and at least one additional active agent selected from at least one corticosteroid, vitamin D analogue (calcipotriene), at least one JAK inhibitor and combinations thereof, the method comprising co-administering to a subject in need thereof therapeutically effective amounts of tapinarof and at least one additional active agent, thereby treating, curing or alleviating the skin disorder. The skin disorder is selected from psoriasis and atopic dermatitis.

In some embodiments, the effective amount is a therapeutically effective amount of tapinarof and at least one additional active agent selected from at least one corticosteroid, calcipotriene, at least one JAK inhibitor and combinations thereof, namely an amount which will cure, treat, mitigate or prevent a skin disorder selected from psoriasis and atopic dermatitis.

In some embodiments, co-administration of tapinarof and at least one additional active agent selected from at least one corticosteroid, calcipotriene, at least one JAK inhibitor and combinations thereof exhibit an additive or synergistic effect while treating or alleviating a skin disorder.

In some other embodiments, the co-administration may be made either by administration of a single combination composition, or alternatively by separate administration of a first composition comprising tapinarof and a second composition comprising the at least one additional active agent selected from at least one corticosteroid, calcipotriene, at least one JAK inhibitor and combinations thereof.

In some embodiment tapinarof and the at least one additional active agent are co-administered as two separate compositions. In some embodiment tapinarof and the at least one additional active agent are co-administered as three separate compositions. In some embodiment tapinarof and the at least one additional active agent are co-administered, wherein each active agent is administered as a separate composition. In some embodiment tapinarof and the at least one additional active agent are administered as a single composition combining tapinarof and the at least one additional active agent.

### Regimen of Administration of the Topical Tapinarof Combination Compositions

Therapeutically effective concentrations of tapinarof and at least one additional active agent in the compositions of this invention, for treatment, prevention or amelioration of the symptoms manifested by the skin disorder are determined by empirical methods known in the art.

Tapinarof and the at least one additional active agent in the combination compositions are included in an amount effective for reducing the skin disorder for which treatment is contemplated. The concentration of the active agents in the composition will depend on absorption, inactivation, excretion rates of the active compound, the synergistic or additive effects, the dosage schedule, and amount administered as well as other factors known to those of skill in the art. Generally, the dosages and concentrations will be lower, typically at least about or at 5 to 10% lower but up to about or at 15, 20, 25, 30, 35, 40, 50, 90 or 95% lower than the amount of tapinarof and/or the at least one additional active agent in the marketed drug currently administered for the treatment of the contemplated skin disorder. The dosage and regimen of administration may be determined by dose finding studies, as known in the art.

Exemplary dosages, strengths and concentrations of tapinarof in the tapinarof combination compositions administered topically, can be in the range of between 0.25-5% w/w or between 0.25-2% w/w or at 0.1%, 0.25%, 0.5%, 1%, 2% , 3%, 4% or 5% w/w.

Typical strengths in the topical combination compositions of this invention are 0.25%, 0.5% or 1% w/w tapinarof.

The frequency of administration can be determined empirically. Exemplary frequencies are once daily, twice daily, weekly, bi-weekly or monthly.

Typical administration frequencies of the topical combination compositions of this invention are once daily and twice daily.

Dosage frequencies can be gradually attenuated over time and maintained at a steady dose suitable for long-term—six months, 1 year, 5 years, 10 years or more, up to lifelong administration to control the symptoms of the skin disorder. For example, dosage administration can begin at from twice a day, to once a day, to two times a week, to once a week, to once every two weeks or less frequent than once every two weeks.

### Treatment of Psoriasis with Tapinarof Combinations

According to one aspect of this invention, psoriasis is treated with tapinarof combinations with at least one corticosteroid and/or calcipotriene.

In another embodiment, psoriasis is treated with between 0.25-5% w/w tapinarof with at least one corticosteroid and/or calcipotriene. In another embodiment, psoriasis is treated with between 0.25-2% w/w tapinarof with at least one corticosteroid and/or calcipotriene.

The corticosteroid used is of very high or high potency (Class 1 or 2, see above). Alternatively, the corticosteroid is selected from a lower group of potency (Class 3-7), which has milder side-effects and lower risk of pituitary suppression.

Due to the synergistic or additive effect with tapinarof, the corticosteroid can also be used at a lower strength (steroid-sparing) than the US-marketed topical drugs used for the treatment of psoriasis.

According to some embodiments, there is provided a method of treatment, prevention or amelioration of psoriasis by topical administration to a subject in need thereof of a composition comprising a therapeutically effective dose of tapinarof and a therapeutically effective dose of at least one additional active agent selected from at least one superpotent (Class 1) or potent (Class 2) corticosteroid, calcipotriene and combinations thereof and a carrier suitable for topical administration.

According to some embodiments, the above at least one superpotent (Class 1) corticosteroid is selected from clobetasol propionate (0.05%), flurandrenolide (0.05%), betamethasone dipropionate (0.05%), diflorasone diacetate (0.05%), desoxymethasone, and fluocinonide (0.1%) and combinations thereof and the potent (Class 2) corticosteroid is selected from betamethasone dipropionate (0.05%), mometasone furoate (0.1%), diflorasone diacetate (0.05%), halcinonide (0.1%), fluocinonide (0.05%), desoxymethasone (0.05%-0.25%) and combinations thereof.

According to some embodiments, there is provided a method of treatment, prevention or amelioration of psoriasis by topical administration to a subject in need thereof of a composition comprising a therapeutically effective dose of tapinarof and a therapeutically effective dose of at least one additional active agent selected from at least one corticosteroid of mid-strength (Class 4), lower mid-strength (Class 5), mild (Class 6) or least potent (Class 7), calcipotriene, combinations thereof and a carrier suitable for topical administration.

According to some embodiments, there is provided a method of treatment, prevention or amelioration of psoriasis by topical administration to a subject in need thereof of a composition comprising a therapeutically effective dose of tapinarof, a therapeutically effective dose of at least one corticosteroid of mid-strength (Class 4), lower mid-strength (Class 5), mild (Class 6) or least potent (Class 7), a therapeutically effective dose of calcipotriene, combinations thereof and a carrier suitable for topical administration.

According to some embodiments, there is provided a method of treatment of psoriasis by topical administration to a subject in need thereof of a composition comprising a therapeutically effective dose of tapinarof and a therapeutically effective dose of at least one corticosteroid of mid-strength (Class 4), lower mid-strength (Class 5), mild (Class 6) or least potent (Class 7), combinations thereof and a carrier suitable for topical administration.

In some other embodiments, the strength of the at least one corticosteroid in the compositions for the treatment of psoriasis is 25% w/w, 50% w/w or 75% w/w lower that the strength of the FDA-approved corticosteroids (see above FDA-approved strengths in parentheses).

In some embodiments, there is provided a composition for the treatment, prevention or amelioration of psoriasis, comprising 1.0% w/w tapinarof, 0.005% w/w calcipotriene, 0.05% betamethasone dipropionate and a carrier suitable for topical administration.

In some embodiments, there is provided a composition for the treatment, prevention or amelioration of psoriasis, comprising 0.5% tapinarof, 0.0025% calcipotriene, 0.025% betamethasone dipropionate and a carrier suitable for topical administration.

The above composition may be a solution, suspension, emulsion or the like and is formulated as a cream, a gel, an ointment, an emulsion, a solution, an elixir, a lotion, a suspension, a tincture, a paste, a foam, an aerosol, a spray, a patch or any other formulation suitable for topical administration.

According to some embodiments, there is provided a method of treatment, prevention or amelioration of psoriasis by topical administration to a subject in need thereof of a composition comprising a therapeutically effective dose of tapinarof in combination with a therapeutically effective dose of at least one additional active agent selected from of at least one superpotent or potent corticosteroid (Class 1-2), at least one JAK inhibitor and combinations thereof.

According to some embodiments, there is provided a method of treatment, prevention or amelioration of psoriasis by topical administration to a subject in need thereof of a composition comprising between 0.25-5% w/w tapinarof in combination with a therapeutically effective dose of at least one additional active agent selected from of at least one superpotent or potent corticosteroid (Class 1-2), at least one JAK inhibitor and combinations thereof.

According to some embodiments, there is provided a method of treatment, prevention or amelioration of psoriasis by topical administration to a subject in need thereof of a composition comprising between 0.25-2% w/w tapinarof in combination with a therapeutically effective dose of at least one additional active agent selected from of at least one superpotent or potent corticosteroid (Class 1-2), at least one JAK inhibitor and combinations thereof.

In some embodiments, there is provided a method of treatment, prevention or amelioration of psoriasis by topical administration to a subject in need thereof of a composition comprising a therapeutically effective dose of tapinarof, a therapeutically effective dose of at least one superpotent or potent corticosteroid (Class 1-2) and a carrier suitable for topical administration.

In some other embodiments, there is provided a method of treatment, prevention or amelioration of psoriasis by topical administration to a subject in need thereof of a composition comprising a therapeutically effective dose of tapinarof in combination with a therapeutically effective dose of at least one superpotent or potent corticosteroid (Class 1-2), a therapeutically effective dose of a JAK inhibitor and a carrier suitable for topical administration.

### Treatment of Atopic Dermatitis (AD) with Tapinarof Combinations

According to some embodiments, there is provided a method of treatment, prevention or amelioration of atopic dermatitis, comprising the administration of a therapeutically effective dose of tapinarof, a therapeutically effective dose of at least one additional active agent selected from of at least one medium or low potency corticosteroid (Class 4-7), at least one JAK inhibitor, combinations thereof and a carrier suitable for topical administration.

According to some embodiments, there is provided a method of treatment, prevention or amelioration of atopic dermatitis, comprising the administration between 0.25-5% w/w or between 0.25-2% w/w tapinarof, a therapeutically effective dose of at least one additional active agent selected from of at least one medium or low potency corticosteroid (Class 4-7), at least one JAK inhibitor, combinations thereof and a carrier suitable for topical administration.

According to some embodiments, there is provided a method of treatment, prevention or amelioration of atopic dermatitis, comprising the administration of a therapeutically effective dose of tapinarof, a therapeutically effective dose of at least one medium or low potency corticosteroid (Class 4-7) and a carrier suitable for topical administration.

According to some embodiments, there is provided a method of treatment, prevention or amelioration of atopic dermatitis, comprising the administration between 0.25-5% w/w or 0.25-2% w/w tapinarof, a therapeutically effective dose of at least one medium or low potency corticosteroid (Class 4-7) and a carrier suitable for topical administration.

According to some embodiments, there is provided a method of treatment, prevention or amelioration of atopic dermatitis, comprising the administration of a therapeutically effective dose of tapinarof, a therapeutically effective dose of at least one JAK inhibitor and a carrier suitable for topical administration.

According to some embodiments, there is provided a method of treatment, prevention or amelioration of atopic dermatitis, comprising the administration between 0.25-5% w/w or 0.25-2% w/w tapinarof, a therapeutically effective dose of at least one medium or low potency corticosteroid (Class 4-7) and a carrier suitable for topical administration.

According to some embodiments, there is provided a method of treatment, prevention or amelioration of atopic dermatitis, comprising the administration of a therapeutically effective dose of tapinarof, a therapeutically effective dose of at least one medium or low potency corticosteroid (Class 4-7), a therapeutically effective dose of a JAK1 or a JAK2 inhibitor and a carrier suitable for topical administration.

According to some embodiments, there is provided a method of treatment, prevention or amelioration of atopic dermatitis, comprising the administration between 0.25-5% w/w or 0.25-2% w/w tapinarof, a therapeutically effective dose of at least one medium or low potency corticosteroid (Class 4-7), a therapeutically effective dose of a JAK1 or a JAK2 inhibitor and a carrier suitable for topical administration.

According to some embodiments, there is provided a method of treatment, prevention or amelioration of atopic dermatitis, comprising the administration of 1% w/w tapinarof, 0.1% w/w triamcinolone, 1.5% w/w ruxolitinib and a carrier suitable for topical administration.

According to some embodiments, there is provided a method of treatment of atopic dermatitis, comprising the administration of 0.5% w/w tapinarof, 0.05% w/w triamcinolone, 0.75% w/w ruxolitinib and a carrier suitable for topical administration.

The above composition may be a solution, suspension, emulsion or the like and is formulated as a cream, a gel, an ointment, an emulsion, a solution, an elixir, a lotion, a suspension, a tincture, a paste, a foam, an aerosol, a spray, a patch or any other formulation suitable for topical administration.

The above Class 4-7 corticosteroids are selected from the following steroids (strengths are indicated in parentheses).

Class 4—mid-strength, comprising 6 steroids: flurandrenolide (0.05%), mometasone furoate (0.1%), triamcinolone acetonide (0.1%), fluocinolone acetonide (0.03%), desoxymethasone (0.05%) and hydrocortisone valerate (0.2%).

Class 5—lower mid-strength, comprising 7 steroids: fluocinolone acetonide (0.01%), flurandrenolide (0.05%), fluticasone propionate (0.05%), prednicarbate (0.1%), desonide (0.05%), hydrocortisone (0.1%), hydrocortisone valerate (0.2%).

Class 6—mild, comprising only 3 steroids: alclomethasone dipropionate (0.05%), fluocinolone acetonide (0.01%), desonide (0.05%),

Class 7—least potent, comprising only one steroid: hydrocortisone (0.5%, 1%, 2%, 2.5%).

### Kits

Kits containing the combination compositions optionally including instructions for administration are provided. The combinations include, for example, the compositions as provided herein, optionally one or more reagents or solutions for diluting the compositions to a desired concentration for administration to a host subject, including human beings. Additionally, provided herein are kits containing the above-described combinations and optionally instructions for administration by topical, transdermal, or other routes, depending on the agent(s) to be delivered.

The compositions provided herein can be packaged as articles of manufacture containing packaging material, a composition provided herein, and a label that indicates that the composition is for treating a skin disorder, such psoriasis or atopic dermatitis, and is formulated for topical or transdermal delivery.

The articles of manufacture provided herein contain packaging materials. Packaging materials for use in packaging pharmaceutical products are well known to those of skill in the art. Examples of pharmaceutical packaging materials include, but are not limited to bottles, tubes, containers, application syringes and any packaging material suitable for a selected formulation and intended mode of administration and treatment.

In some embodiments, there is provided a topical composition comprising from about 0.25% w/w to about 5.0% w/w tapinarof or from about 0.25% w/w to about 2.0% w/w tapinarof and at least one additional active agent, selected from about 0.01% w/w to about 0.25% w/w at least one corticosteroid of potency class 1 or 2 or from about 0.0025% w/w to about 2.5% w/w at least one corticosteroid of potency class 3-7, from about 0.001% w/w to about 0.005% w/w calcipotriene, from about 0.1% w/w to about 3% w/w at least one JAK1, JAK2 or JAK 1/2 inhibitor and combinations thereof, and a carrier suitable for topical administration.

In some other embodiments, there is provided a composition comprising from about 0.25% w/w to about 5.0% w/w tapinarof or from about 0.25% w/w to about 2.0% w/w tapinarof and at least one additional active agent selected from about 0.01% w/w to about 0.25% w/w at least one corticosteroid of potency class 1 or 2 and combinations thereof, and a carrier suitable for topical administration.

According to some embodiments, there is provided a composition comprising from about 0.25% w/w to about 5.0% w/w tapinarof or from about 0.25% w/w to about 2.0% w/w tapinarof and at least one additional active agent selected from about 0.0025% w/w to about 2.5% w/w at least one corticosteroid of potency class 3-7 and combinations thereof, and a carrier suitable for topical administration.

According to some other embodiments, there is provided a composition comprising from about 0.25% w/w to about 5.0% w/w tapinarof or from about 0.25% w/w to about 2.0% w/w tapinarof and at least one additional active agent selected from about 0.01% w/w to about 0.25% w/w at least one corticosteroid of potency class 1 or 2, from about 0.001% w/w to about 0.005% w/w calcipotriene and combinations thereof, and a carrier suitable for topical administration.

In some embodiments, there is provided a composition of comprising from about 0.25% w/w to about 5.0% w/w tapinarof or from about 0.25% w/w to about 2.0% w/w tapinarof and at least one additional active agent selected from about 0.0025% w/w to about 2.5% w/w at least one corticosteroid of potency class 3-7, from about 0.001% w/w to about 0.005% w/w calcipotriene and combinations thereof, and a carrier suitable for topical administration.

In some other embodiments, there is provided a composition comprising from about 0.25% w/w to about 5.0% w/w tapinarof or from about 0.25% w/w to about 2.0% w/w tapinarof and at least one additional active agent selected from about 0.1% w/w to about 3% w/w at least one JAK1, JAK2 or JAK 1/2 inhibitor and combinations thereof, and a carrier suitable for topical administration.

In some embodiments, there is provided a composition comprising from about 0.25% w/w to about 5.0% w/w tapinarof or from about 0.25% w/w to about 2.0% w/w tapinarof, from about 0.0025% w/w to about 2.5% w/w at least one corticosteroid of potency class 3-7, from about 0.1% w/w to about 3% w/w at least one JAK1, JAK2 or JAK 1/2 inhibitor, combinations thereof and a carrier suitable for topical administration.

In some other embodiments, there is provided a dosage form comprising any of the compositions of this invention, wherein formulated in a dosage form selected from a cream, a gel, an ointment, an emulsion, a solution, a suspension, an elixir, a lotion, a tincture, a paste, a foam, an aerosol, a spray, a patch, a transdermal patch and an applicator syringe.

According to some embodiments, there is provided a method of treatment, prevention or alleviation of a skin disorder selected from plaque, guttate, inverse, flexural/inverse pustular or erythrodermic psoriasis and atopic dermatitis, by once daily or twice daily topical administration to a subject in need thereof of any one of the compositions of this invention, wherein the composition is formulated in a dosage form selected from a cream, a gel, an ointment, an emulsion, a solution, a suspension, an elixir, a lotion, a tincture, a paste, a foam, an aerosol, a spray, a patch, a transdermal patch and an applicator syringe.

According to some other embodiments, there is provided a method of treatment, prevention or alleviation of a skin disorder selected from plaque, guttate, inverse, flexural/inverse pustular or erythrodermic psoriasis, by once daily or twice daily topical administration to a subject in need thereof a composition comprising from about 0.25% w/w to about 5.0% w/w tapinarof or from about 0.25% w/w to about 2.0% w/w tapinarof and at least one additional active agent selected from about 0.01% w/w to about 0.25% w/w at least one corticosteroid of potency class 1 or 2 and combinations thereof and a carrier suitable for topical administration, wherein the composition is formulated in a dosage form selected from a cream, a gel, an ointment, an emulsion, a solution, a suspension, an elixir, a lotion, a tincture, a paste, a foam, an aerosol, a spray, a patch, a transdermal patch and an applicator syringe.

In some embodiments, there is provided a method of treatment, prevention or alleviation of a skin disorder selected from plaque, guttate, inverse, flexural/inverse, pustular or erythrodermic psoriasis, by once daily or twice daily topical administration to a subject in need thereof a composition comprising from about 0.25% w/w to about 5.0% w/w tapinarof or from 0.25% w/w to about 2.0% w/w tapinarof, from about 0.0025% w/w to about 2.5% w/w at least one corticosteroid of potency class 3-7 and combinations thereof and a carrier suitable for topical administration, wherein the composition is formulated in a dosage form selected from a cream, a gel, an ointment, an emulsion, a solution, a suspension, an elixir, a lotion, a tincture, a paste, a foam, an aerosol, a spray, a patch, a transdermal patch and an applicator syringe.

In some other embodiments, there is provided a method of treatment, prevention or alleviation of a skin disorder selected from plaque, guttate, inverse, flexural/inverse, pustular or erythrodermic psoriasis, by once daily or twice daily topical administration to a subject in need thereof a composition comprising from about 0.25% w/w to about 5.0% w/w tapinarof or from about 0.25% w/w to about 2.0% w/w tapinarof, from about 0.01% w/w to about 0.25% w/w at least one corticosteroid of potency class 1 or 2, from about 0.001% w/w to about 0.005% w/w calcipotriene and combinations thereof and a carrier suitable for topical administration, wherein the composition is formulated in a dosage form selected from a cream, a gel, an ointment, an emulsion, a solution, a suspension, an elixir, a lotion, a tincture, a paste, a foam, an aerosol, a spray, a patch, a transdermal patch and an applicator syringe.

According to some embodiments, there is provided a method of treatment, prevention or alleviation of a skin disorder selected from plaque, guttate, inverse, flexural/inverse, pustular or erythrodermic psoriasis, by once daily or twice daily topical administration to a subject in need thereof a composition comprising from about 0.25% w/w to about 5.0% w/w tapinarof or from about 0.25% w/w to about 2.0% w/w tapinarof, from about 0.0025% w/w to about 2.5% w/w at least one corticosteroid of potency class 3-7, from about 0.001% w/w to about 0.005% w/w calcipotriene and combinations thereof and a carrier suitable for topical administration, wherein the composition is formulated in a dosage form selected from a cream, a gel, an ointment, an emulsion, a solution, a suspension, an elixir, a lotion, a tincture, a paste, a foam, an aerosol, a spray, a patch, a transdermal patch and an applicator syringe.

According to some other embodiments, there is provided a method of treatment, prevention or alleviation of atopic dermatitis, by once daily or twice daily topical administration to a subject in need thereof a composition comprising from about 0.25% w/w to about 5.0% w/w tapinarof or from about 0.25% w/w to about 2.0% w/w tapinarof, from about 0.0025% w/w to about 2.5% w/w at least one corticosteroid of potency class 3-7, combinations thereof, and a carrier suitable for topical administration, wherein the composition is formulated in a dosage form selected from a cream, a gel, an ointment, an emulsion, a solution, a suspension, an elixir, a lotion, a tincture, a paste, a foam, an aerosol, a spray, a patch, a transdermal patch and an applicator syringe.

In some embodiments, there is provided a method of treatment, prevention or alleviation of atopic dermatitis, by once daily or twice daily topical administration to a subject in need thereof a composition comprising from about 0.25% w/w to about 5.0% w/w tapinarof or from about 0.25% w/w to about 2.0% w/w tapinarof, from about 0.1% w/w to about 3% w/w at least one JAK1, JAK2 or JAK 1/2 inhibitor and combinations thereof, and a carrier suitable for topical administration, wherein the composition is formulated in a dosage form selected from a cream, a gel, an ointment, an emulsion, a solution, a suspension, an elixir, a lotion, a tincture, a paste, a foam, an aerosol, a spray, a patch, a transdermal patch and an applicator syringe.

In some other embodiments, there is provided a method of treatment, prevention or alleviation of atopic dermatitis, by once daily or twice daily topical administration to a subject in need thereof of a composition comprising from about 0.25% w/w to about 5.0% w/w tapinarof or from about 0.25% w/w to about 2.0% w/w tapinarof, from about 0.0025% w/w to about 2.5% w/w at least one corticosteroid of potency class 3-7, from about 0.1% w/w to about 3% w/w at least one JAK1, JAK2 or JAK 1/2 inhibitor and combinations thereof, and a carrier suitable for topical administration, wherein the composition is formulated in a dosage form selected from a cream, a gel, an ointment, an emulsion, a solution, a suspension, an elixir, a lotion, a tincture, a paste, a foam, an aerosol, a spray, a patch, a transdermal patch and an applicator syringe.

According to some embodiments, there is provided a regimen of administration comprising the once daily or twice daily administration to a patient in need thereof of a dosage form comprising any one of the compositions of this invention, wherein the composition is formulated in a dosage form selected from a cream, a gel, an ointment, an emulsion, a solution, a suspension, an elixir, a lotion, a tincture, a paste, a foam, an aerosol, a spray, a patch, a transdermal patch or an applicator syringe, until remission or according to doctor's orders.

According to some other embodiments, there is provided a kit comprising at least one dosage form comprising any one of the compositions of this invention, wherein the composition is formulated in a dosage form selected from a cream, a gel, an ointment, an emulsion, a solution, a suspension, an elixir, a lotion, a tincture, a paste, a foam, an aerosol, a spray, a patch, a transdermal patch or an applicator syringe, and instructions for use.

### DEFINITIONS

Unless defined otherwise, all technical and scientific terms used herein have the same meaning as is commonly understood by one of skill in the art to which the invention pertains. In case of conflict, the specification, including definitions, takes precedence. All patents, patent applications, published applications, articles, publications and other published materials referred to throughout the entire disclosure herein, unless noted otherwise, are incorporated by reference in their entirety.

As used herein, the indefinite articles “a” and “an” mean “at least one” or “one or more” unless the context clearly dictates otherwise.

As used herein, the term “calcipotriene” as used herein refers to calcipotriene or to hydrate thereof.

As used herein, the term “treating” or” treatment” includes curing a condition, treating a condition, preventing a condition, treating symptoms of a condition, curing symptoms of a condition, ameliorating symptoms of a condition, treating effects of a condition, ameliorating effects of a condition, and preventing results of a condition.

As used herein, the terms “pharmaceutically active agent” or “active agent” or “active pharmaceutical ingredient” or “API” are interchangeable and mean the ingredient is a pharmaceutical drug which is biological active and is regulatory approved or approvable as such.

The term “ingredient” refers to a pharmaceutically acceptable ingredient which is included or is amenable to be included in FDA's Inactive Ingredient database (IIG). Inactive ingredients sometimes exhibit some therapeutic effects, although they are not drugs.

As used herein, a “pharmaceutical composition” refers to a composition comprising one or more active ingredients with other components such as pharmaceutically acceptable ingredients or excipients. The purpose of a pharmaceutical composition is to facilitate administration of an active ingredient to a subject.

As used herein, the term “essentially free” generally refers to a composition having less than about 2 percent by weight, more preferably 1 percent per weight, less than about 0.5 percent by weight or even less than 0.1 percent by weight of a certain ingredient, based on the total weight of the composition.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “10 μm” is intended to mean “about 10 μm”.

As used herein, numerical ranges preceded by the term “about” should not be considered to be limited to the recited range. Rather, numerical ranges preceded by the term “about” should be understood to include a range accepted by those skilled in the art for any given element in formulations according to the present invention.

As used herein, when a numerical value is preceded by the term “about”, the term “about” is intended to indicate +/−10%.

The terms “comprise”, “comprising”, “includes”, “including”, “having” and their conjugates mean “including but not limited to”.

The term “consisting of” means “including and limited to”.

The term “consisting essentially of” means that the composition, method formulation may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment.

Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein and the laboratory procedures utilized in the present invention include chemical, molecular and biochemical, techniques. Such techniques are thoroughly explained in the literature. General references are provided throughout this document. The procedures therein are believed to be well known in the art and are provided for the convenience of the reader. All the information contained therein is incorporated herein by reference.

### Example 1

### Preparation of a Tapinarof/Betamethasone Dipropionate Cream Composition for the Treatment of Psoriasis

The topical tapinarof/betamethasone dipropionate combination cream consists of:

0.25-2.0% w/w tapinarof,

0.01-0.05% w/w betamethasone dipropionate,

0.1-0.5% w/w menthol,

0.01-0.05% w/w butylated hydroxyanisole (BHA),

15-30% w/w propylene glycol,

5.0-15.0% polysorbate 80,

10-25% w/w glyceryl monostearate,

10-25% w/w of thickener octadecanol,

6.0-7.0% of 0.1M NaOH or HCl as an aqueous phase pH,

Make up to 100% with purified water and

Adjust the pH to 6.0-7.0 with 0.1 M NaOH or HCl

The cream composition is prepared by the following steps:

(1) weigh tapinarof having an average particle size of less than 1 μm;

(2) heat the propylene glycol to 60° C. in a water bath;

(3) add to the heated propylene glycol while stirring tapinarof, betamethasone dipropionate, BHT, menthol, octadecanol, polysorbate 80 and glyceryl monostearate, and dissolve to obtain an oil phase;

(4) prepare the aqueous phase by heating purified water in a water bath to 60° C., stir in and dissolve polysorbate 80 and adjust the pH to 6.0-7.0 with 0.1 M NaOH or HCl;

(5) add the aqueous phase to the oil phase under vacuum stirring, and cool to room temperature to obtain a cream;

(6) fill the tapinarof/betamethasone dipropionate combination cream in an aluminum tube or other delivery system.

### Example 2

### Preparation of a Tapinarof/Calcipotriene Cream Composition for the Treatment of Psoriasis

The topical tapinarof/calcipotriene combination cream consists of:

0.25-2.0% w/w tapinarof,

0.001-0.005% w/w calcipotriene or calcipotriene hydrate (calculated as calcipotriene),

0.1-0.5% w/w menthol,

0.01-0.05% w/w butylated hydroxyanisole (BHA),

15-30% w/w propylene glycol,

5.0-15.0% polysorbate 80,

10-25% w/w glyceryl monostearate,

10-25% w/w of thickener octadecanol,

6.0-7.0% of 0.1M NaOH or HCl as an aqueous phase pH,

Make up to 100% with purified water and p Adjust the pH to 6.0-7.0 with 0.1 M NaOH or HCl

The cream composition is prepared by the following steps:

(1) weigh tapinarof having an average particle size of less than 1 μm;

(2) heat the propylene glycol to 60° C. in a water bath;

(3) add to the heated propylene glycol while stirring tapinarof, calcipotriene, BHT, menthol, octadecanol, polysorbate 80 and glyceryl monostearate, and dissolve to obtain an oil phase;

(4) prepare the aqueous phase by heating purified water in a water bath to 60° C., stir in and dissolve polysorbate 80 and adjust the pH to 6.0-7.0 with 0.1 M NaOH or HCl;

(5) add the aqueous phase to the oil phase under vacuum stirring, and cool to room temperature to obtain a cream;

(6) fill the tapinarof/calcipotriene combination cream in an aluminum tube or other delivery system.

### Example 3

### Preparation of a Tapinarof/Calcipotriene/Betamethasone Dipropionate Cream Composition for the Treatment of Psoriasis

The topical tapinarof/calcipotriene/betamethasone dipropionate combination cream consists of:

0.25-2.0% w/w tapinarof,

0.001-0.005% w/w calcipotriene or calcipotriene hydrate (calculated as calcipotriene),

0.01-0.05% w/w betamethasone dipropionate,

0.1-0.5% w/w menthol,

0.01-0.05% w/w butylated hydroxyanisole (BHA),

15-30% w/w propylene glycol,

5.0-15.0% polysorbate 80,

10-25% w/w glyceryl monostearate,

10-25% w/w of thickener octadecanol,

6.0-7.0% of 0.1M NaOH or HCl as an aqueous phase pH,

Make up to 100% with purified water and

Adjust the pH to 6.0-7.0 with 0.1 M NaOH or HCl

The cream composition is prepared by the following steps:

(1) weigh tapinarof having an average particle size of less than 1 μm;

(2) heat the propylene glycol to 60° C. in a water bath;

(3) add to the heated propylene glycol while stirring tapinarof, calcipotriene, betamethasone dipropionate, BHT, menthol, octadecanol, polysorbate 80 and glyceryl monostearate, and dissolve to obtain an oil phase;

(4) prepare the aqueous phase by heating purified water in a water bath to 60° C., stir in and dissolve polysorbate 80 and adjust the pH to 6.0-7.0 with 0.1 M NaOH or HCl;

(5) add the aqueous phase to the oil phase under vacuum stirring, and cool to room temperature to obtain a cream;

(6) fill the tapinarof/calcipotriene/betamethasone dipropionate combination cream in an aluminum tube or other delivery system.

### Example 4

### Preparation of a Tapinarof/Triamcinolone Cream Composition for the Treatment of Atopic Dermatitis

The topical tapinarof/triamcinolone combination cream consists of:

0.25-2.0% w/w tapinarof,

0.05-0.2% w/w triamcinolone,

0.1-0.5% w/w menthol,

0.01-0.05% w/w butylated hydroxyanisole (BHA),

15-30% w/w propylene glycol,

5.0-15.0% polysorbate 80,

10-25% w/w glyceryl monostearate,

10-25% w/w of thickener octadecanol,

6.0-7.0% of 0.1M NaOH or HCl for adjusting the pH to 6.0-7.0

Purified water up to 100%

The cream composition is prepared by the following steps:

(1) weigh tapinarof having an average particle size of less than 1 μm;

(2) heat the propylene glycol to 60° C. in a water bath;

(3) add to the heated propylene glycol while stirring tapinarof, triamcinolone, BHT, menthol, octadecanol, polysorbate 80 and glyceryl monostearate, and dissolve to obtain an oil phase;

(4) prepare the aqueous phase by heating purified water in a water bath to 60° C., stir in and dissolve polysorbate 80 and adjust the pH to 6.0-7.0 with 0.1 M NaOH or HCl;

(5) add the aqueous phase to the oil phase under vacuum stirring, and cool to room temperature to obtain a cream;

(6) fill the tapinarof/triamcinolone combination cream in an aluminum tube or other delivery system.

### Example 5

### Preparation of a Tapinarof/Ruxolitinib Cream Composition for the Treatment of Atopic Dermatitis

The topical tapinarof/ruxolitinib combination cream consists of:

0.25-2.0% w/w tapinarof,

1-2% w/w ruxolitinib phosphate (calculated as base),

0.1-0.5% w/w menthol,

0.01-0.05% w/w butylated hydroxyanisole (BHA),

15-30% w/w propylene glycol,

5.0-15.0% polysorbate 80,

10-25% w/w glyceryl monostearate,

10-25% w/w of thickener octadecanol,

6.0-7.0% of 0.1M NaOH or HCl for adjusting the pH to 6.0-7.0

Purified water up to 100%

The cream composition is prepared by the following steps:

(1) weigh tapinarof having an average particle size of less than 1 μm;

(2) heat the propylene glycol to 60° C. in a water bath;

(3) add to the heated propylene glycol while stirring tapinarof, ruxolitinib phosphate, BHT, menthol, octadecanol, polysorbate 80 and glyceryl monostearate, and dissolve to obtain an oil phase;

(4) prepare the aqueous phase by heating purified water in a water bath to 60° C., stir in and dissolve polysorbate 80 and adjust the pH to 6.0-7.0 with 0.1 M NaOH or HCl;

(5) add the aqueous phase to the oil phase under vacuum stirring, and cool to room temperature to obtain a cream;

(6) fill the tapinarof/ruxolitinib combination cream in an aluminum tube or other delivery system.

### Example 6

### Preparation of a Tapinarof/Triamcinolone/Ruxolitinib Cream Composition for the Treatment of Atopic Dermatitis

The topical tapinarof/triamcinolone/ruxolitinib combination cream consists of:

0.25-2.0% w/w tapinarof,

0.05-0.2% w/w triamcinolone,

1-2% w/w ruxolitinib phosphate (calculated as base),

0.1-0.5% w/w menthol,

0.01-0.05% w/w butylated hydroxyanisole (BHA),

15-30% w/w propylene glycol,

5.0-15.0% polysorbate 80,

10-25% w/w glyceryl monostearate,

10-25% w/w of thickener octadecanol,

6.0-7.0% of 0.1M NaOH or HCl for adjusting the pH to 6.0-7.0

Purified water up to 100%

The cream composition is prepared by the following steps:

(1) weigh tapinarof having an average particle size of less than 1 μm;

(2) heat the propylene glycol to 60° C. in a water bath;

(3) add to the heated propylene glycol while stirring tapinarof, triamcinolone, ruxolitinib phosphate, BHT, menthol, octadecanol, polysorbate 80 and glyceryl monostearate, and dissolve to obtain an oil phase;

(4) prepare the aqueous phase by heating purified water in a water bath to 60° C., stir in and dissolve polysorbate 80 and adjust the pH to 6.0-7.0 with 0.1 M NaOH or HCl;

(5) add the aqueous phase to the oil phase under vacuum stirring, and cool to room temperature to obtain a cream;

(6) fill the tapinarof/triamcinolone/ruxolitinib combination cream in an aluminum tube or other delivery system.

### Example 7

### Preparation of Tapinarof, 1% Lotion

| Raw Material (compendial Name) | % w/w |
|---|---|
| Tapinarof 98% | 1.0 |
| Castor oil | 4.0 |
| Mineral oil light | 4.0 |
| Diethylene glycol monoethyl ether | 5.5 |
| Dimethyl Sulfoxide | 5.5 |
| Sorbitan Monooleate | 0.1 |
| Propylene glycol | 10.0 |
| Disodium Edetate (EDTA) | 0.1 |
| Carbomer Copolymer Type B Pemulen ®TR-1 | 0.4 |
| Carbomer Homopolymer Type A Carbopol ®981 | 0.6 |
| Purified water | 64.00 |
| Benzoic Acid | 0.25 |
| BHT | 0.1 |
| Citric Acid | 0.1 |
| Sodium Citrate | 0.2 |
| Sodium hydroxide pellets | For pH adjustment |
| Purified water | q.s. to 100 |

### Water Phase

Into a glass beaker water and Benzoic Acid were added. The beaker was placed inside a hot water bath adjusted to 60° C. and the mixture was mixed with a magnetic stirrer until a clear solution free from particles was obtained. Then EDTA, Citric Acid and Sodium Citrate were added. The mixing was continued until a clear solution was obtained. The solution was cooled down to room temperature. Then, the pH was slowly adjusted to pH 6.0 with NaOH 20%.

### Oil Phase

In a separate glass beaker Mineral oil light, castor oil, span 80 and BHT were weighed. The beaker was placed inside a hot water bath adjusted to 60° C. and the mixture was mixed with a magnetic stirrer until a uniform solution was obtained. Then Carbopol® 981 and Pemulen® TR-1 were slowly added and the mixing was continued until a homogenous mixture was obtained. The mixture was cooled down to room temperature.

### Active Phase

Into a separate glass beaker Propylene Glycol, Transcutol and DMSO were weighed. The mixture was mixed with a magnetic stirrer until a uniform homogenous solution was obtained. The beaker was covered with an aluminum foil and placed in a yellow light hood. Tapinarof was slowly added, and the mixing was continued for about 1 h until a clear solution free from particles was obtained.

The oil phase was slowly added to the water phase while homogenizing for about 5 minutes, until there were no lumps. Then, the active phase was slowly added to the Water+Oil phase while homogenizing for about 5 minutes.

Water was added for batch completion, and final pH was measured to conform it is around pH 5.

1. A topical composition comprising from about 0.25% w/w to about 2.0% w/w tapinarof and at least one additional active agent, selected from about 0.01% w/w to about 0.25% w/w at least one corticosteroid of potency class 1 or 2 or from about 0.0025% w/w to about 2.5% w/w at least one corticosteroid of potency class 3-7, from about 0.001% w/w to about 0.005% w/w calcipotriene, from about 0.1% w/w to about 3% w/w at least one JAK1, JAK2 or JAK 1/2 inhibitor and combinations thereof, and a carrier suitable for topical administration.

2. The composition of claim 1, wherein the at least one additional active agent is selected from about 0.01% w/w to about 0.25% w/w at least one corticosteroid of potency class 1 or 2 and combinations thereof.

3. The composition of claim 1, wherein the at least one additional active agent is selected from about 0.0025% w/w to about 2.5% w/w at least one corticosteroid of potency class 3-7 and combinations thereof.

4. The composition of claim 1, wherein the at least one additional active agent is selected from about 0.01% w/w to about 0.25% w/w at least one corticosteroid of potency class 1 or 2, from about 0.001% w/w to about 0.005% w/w calcipotriene or hydrate thereof and combinations thereof.

5. The composition of claim 1, wherein the at least one additional active agent is selected from about 0.0025% w/w to about 2.5% w/w at least one corticosteroid of potency class 3-7, from about 0.001% w/w to about 0.005% w/w calcipotriene and combinations thereof.

6. The composition of claim 1, wherein the at least one additional active agent is selected from about 0.1% w/w to about 3% w/w at least one JAK1, JAK2 or JAK 1/2 inhibitor and combinations thereof.

7. The composition of claim 3, wherein further comprising from about 0.1% w/w to about 3% w/w at least one JAK1, JAK2 or JAK 1/2 inhibitor and combinations thereof.

8. A dosage form comprising the composition of claim 1, wherein the composition is formulated in a dosage form selected from a cream, a gel, an ointment, an emulsion, a solution, a suspension, an elixir, a lotion, a tincture, a paste, a foam, an aerosol, a spray, a patch, a transdermal patch and an applicator syringe.

9. A method of treatment, prevention or alleviation of a skin disorder selected from plaque, guttate, flexural/inverse, pustular or erythrodermic psoriasis and atopic dermatitis, by once daily or twice daily topical administration to a subject in need thereof of one of the compositions of claim 1, wherein the composition is formulated in a dosage form selected from a cream, a gel, an ointment, an emulsion, a solution, a suspension, an elixir, a lotion, a tincture, a paste, a foam, an aerosol, a spray, a patch, a transdermal patch and an applicator syringe.

10. The method of claim 9, wherein the treatment comprises once daily or twice daily topical administration to a subject in need thereof a topical composition comprising from about 0.25% w/w to about 2.0% w/w tapinarof and from about 0.01% w/w to about 0.25% w/w at least one corticosteroid of potency class 1 or 2 or combination thereof and a carrier suitable for topical administration and the skin disorder is selected from plaque, guttate, flexural/inverse, pustular or erythrodermic psoriasis and combinations thereof.

11. The method of claim 9, wherein the treatment comprises once daily or twice daily topical administration to a subject in need thereof a topical composition comprising from about 0.25% w/w to about 2.0% w/w tapinarof and from about 0.0025% w/w to about 2.5% w/w at least one corticosteroid of potency class 3-7 or combinations thereof and a carrier suitable for topical administration and the skin disorder is selected from plaque, guttate, flexural/inverse, pustular or erythrodermic psoriasis and combinations thereof.

12. The method of claim 9, wherein the treatment comprises once daily or twice daily topical administration to a subject in need thereof a topical composition comprising from about 0.25% w/w to about 2.0% w/w tapinarof and from about 0.01% w/w to about 0.25% w/w at least one corticosteroid of potency class 1 or 2, or from about 0.001% w/w to about 0.005% w/w calcipotriene or hydrate thereof or combinations thereof and a carrier suitable for topical administration and the skin disorder is selected from plaque, guttate, flexural/inverse, pustular or erythrodermic psoriasis and combinations thereof.

13. The method of claim 9, wherein the treatment comprises once daily or twice daily topical administration to a subject in need thereof a topical composition comprising from about 0.25% w/w to about 2.0% w/w tapinarof and from about 0.0025% w/w to about 2.5% w/w at least one corticosteroid of potency class 3-7, or from about 0.001% w/w to about 0.005% w/w calcipotriene or combinations thereof and a carrier suitable for topical administration and the skin disorder is selected from plaque, guttate, flexural/inverse, pustular or erythrodermic psoriasis and combinations thereof.

14. The method of claim 9, wherein the treatment comprises once daily or twice daily topical administration to a subject in need thereof a topical composition comprising from about 0.25% w/w to about 2.0% w/w tapinarof and from about 0.0025% w/w to about 2.5% w/w at least one corticosteroid of potency class 3-7 or combinations thereof and a carrier suitable for topical administration and the skin disorder is atopic dermatitis.

15. The method of claim 9, wherein the treatment comprises once daily or twice daily topical administration to a subject in need thereof a topical composition comprising from about 0.25% w/w to about 2.0% w/w tapinarof and from about 0.1% w/w to about 3% w/w at least one JAK1, JAK2 or JAK 1/2 inhibitor or combinations thereof and a carrier suitable for topical administration and the skin disorder is atopic dermatitis.

16. The method of claim 9, wherein the treatment comprises once daily or twice daily topical administration to a subject in need thereof a topical composition comprising from about 0.25% w/w to about 2.0% w/w tapinarof and from about 0.0025% w/w to about 2.5% w/w at least one corticosteroid of potency class 3-7 and from about 0.1% w/w to about 3% w/w at least one JAK1, JAK2 or JAK 1/2 inhibitor or combinations thereof and a carrier suitable for topical administration and the skin disorder is atopic dermatitis.

17. A regimen of administration comprising the once daily or twice daily administration a patient in need thereof of the dosage form of claim 8 to until complete remission or according to doctor's orders.

18. A kit comprising one or more dosage forms of claim 8 and instructions for use.
